(19) United States
(12) Patent Application Publication
(10) Pub. No.: US 2025/0387534 A1
(43) Pub. Date: Dec. 25, 2025

(54) METHOD TO DIRECT VASCULARIZATION OF TISSUE GRAFTS

(71) Applicant: The Regents of the University of California, Oakland, CA (US)

(72) Inventors: Eric KURZROCK, Sacramento, CA (US); Stephanie OSBORN, Southborough, MA (US)

(21) Appl. No.: 18/880,497

(22) PCT Filed: Jun. 29, 2023
(86) PCT No.: PCT/US2023/026550

§ 371 Date: Dec. 31, 2024

Related U.S. Application Data
(60) Provisional application No. 63/367,535, filed on Jul. 1, 2022.

(51) Int. Cl. A61L 27/36; A61L 27/18; A61L 27/56

(52) U.S. Cl. CPC A61L 27/3604; A61L 27/18; A61L 27/56

(57) ABSTRACT

The disclosure provides compositions and methods for directing the growth of blood vessels in a tissue graft. The compositions and methods provide the advantage of directing the growth, size and pattern of blood vessels from the periphery of the graft, which recapitulates or improves the native blood vessel orientation in the host tissue. The compositions comprise a tissue graft that comprises a matrix attached to a barrier. The barrier comprises a porous membrane that permits passage of water and nutrients but blocks blood vessel growth. The methods comprise implanting the tissue graft on a host tissue in a subject and incubating the graft on the host tissue such that blood vessels grow around the edges (periphery) of the barrier or through intentional internal openings in the barrier and into the matrix. The methods can further comprise transplanting the vascularized tissue graft to a second location in the subject. Thus, the methods can be used in a staged implant procedure to create autologous, vascularized bioengineered tissue grafts.

### CROSS-REFERENCE TO RELATED APPLICATIONS

This application claims priority to U.S. Provisional Application No. 63/367,535, filed Jul. 1, 2022, the disclosure of which is herein incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. W81XWH-18-1-0657, awarded by the U.S. Army. The Government has certain rights in the invention.

### BACKGROUND

Prior investigations have shown that implantation of grafts with endothelial cells, stem cells and/or growth factors enhances endothelial counts, often referred to as mean vessel density (MVD).^{5,20-22} These studies were limited to small animal models where survival was likely due to small graft size, which allows ingrowth of host vessels and imbibition, passive absorption of fluids. Unfortunately, imbibition cannot reach the center of large grafts and regrowth of vessels is not timely enough (average 0.25 mm/day) to support perfusion and thereby prevent ischemia and contraction. Indeed, studies have shown that merely seeding a graft with endothelial cells does not produce an organized or functional vascular network in vitro. In contrast to solid organ grafts, bioengineered bladder has already been tested in human clinical trials but failed due to a lack of vessels. The instant disclosure provides vascularized tissue grafts that may be useful for clinical applications.

### BRIEF SUMMARY

The present disclosure provides compositions and methods related to directing the growth of blood vessels in a tissue graft, and methods for producing a vascularized tissue graft.

In one aspect, the disclosure provides a method for directing the growth of blood vessels in a tissue graft, the method comprising the steps of: i) implanting a graft on a tissue in a host organism, wherein the graft comprises a matrix attached to a porous barrier and the porous barrier is positioned between the host tissue and the matrix; and ii) incubating the graft in the host, wherein blood vessels grow around the edges of the barrier, thereby directing the growth of blood vessels around the barrier and into the matrix.

In another aspect, the disclosure provides a method for producing a vascularized tissue graft, the method comprising the steps of i) implanting a graft on a tissue in a host organism, wherein the graft comprises a matrix attached to a barrier and the barrier is positioned between the host tissue and the matrix; and ii) incubating the graft in the host such that blood vessels grow around the edges of the barrier and have an increased longitudinal index in the center of the tissue graft compared to tissue grafts without a barrier.

In some embodiments, the barrier comprises a porous membrane that permits passage of water and nutrients but blocks blood vessel growth. In some embodiments, the porous membrane comprises a pore size from about 0 microns to about 5 microns. In some embodiments, the porous membrane comprises a pore size of about 0.4 microns.

In some embodiments, the porous membrane is a polyester membrane. In some embodiments, the porous membrane is a polycarbonate membrane. In some embodiments, wherein the barrier comprises a single contiguous porous membrane.

In some embodiments, the barrier comprises a non-porous biocompatible stabilizing mesh or frame attached to the porous membrane. In some embodiments, the stabilizing mesh or frame is about 1 mm to about 2 mm thick. In some embodiments, the stabilizing mesh or frame comprises silicone.

In some embodiments, the barrier comprises one or more openings that permit blood vessel growth through the barrier.

In some embodiments, the host tissue is selected from a muscle, subcutaneous fat, or a kidney capsule. In some embodiments, the muscle is a rectus abdominis muscle.

In some embodiments, the matrix comprises decellularized tissue. In some embodiments, the decellularized tissue is selected from bladder, kidney, liver, heart, lung, pancreas, connective tissue, bone, epidermis, or dermis. In some embodiments, the matrix comprises decellularized urinary bladder matrix (UBM).

In some embodiments, the matrix is a synthetic matrix.

In some embodiments, the graft size is about 25 cm² to about 300 cm².

In some embodiments, the graft is implanted in the host tissue for a period of about 2 weeks to about 6 months.

In some embodiments, the average length of coronal blood vessels is increased compared to grafts without a barrier. In some embodiments, the ratio of coronal (long) to transverse (short) vessels is increased compared to grafts without a barrier. In some embodiments, the longitudinal index (Li) of blood vessels is increased compared to grafts without a barrier. In some embodiments, the mean vessel density (MVD) is the same or substantially similar between grafts equal to or greater than 100 cm² that are implanted for about 6 months with and without a porous barrier.

In another aspect, the method further comprises transplanting the graft to a second host tissue after a period of time sufficient for blood vessel growth into the matrix. In some embodiments, the second host tissue is selected from heart, kidney, urinary bladder, liver, gastrointestinal tract tissues such as stomach, small intestine, or large intestine, pancreas, lung, and dermal or epidermal tissue. In some embodiments, the second host tissue is bladder tissue. In some embodiments the period of time is from about 2 weeks to about 6 months.

In another aspect, the disclosure provides a tissue graft comprising a matrix described herein attached to a barrier, the barrier comprising a porous membrane described herein, and the matrix comprising a decellularized tissue described herein.

### BRIEF DESCRIPTION OF THE DRAWINGS

FIGS. 1A and 1B show a representative method for growing blood vessels that are long and coronally-oriented from the anastomosis toward the central portion of the graft. FIG. 1A shows angiogenesis of host vessels (dark grey) into a graft with pre-existing vessels (light grey) leads to inosculation, which establishes timely blood supply. Asterisk (*) represents central most part of graft. FIG. 1B shows vessel patterns in the presence or absence of a centrally placed barrier. A barrier induces long, coronally-oriented blood vessels from the graft periphery, the future site of anastomosis to the bladder.

FIGS. 2A-2D show a centrally placed barrier directs vascularization in the rat rectus implant model. FIG. 2A shows H&E staining of full grafts recovered at various time-points and cut in the coronal plane (2× mag stitched map); representative images (20× mag) of middle (M) and peripheral (P) areas of UBMs with and without a barrier. FIG. 2B shows MVD at periphery and in middle of grafts implanted with and without barrier. FIG. 2C shows the length of CD31+vessels were measured in ImageJ at 2 weeks and evaluated by 3 indices: the average length of coronal (long) vessels, the ratio of coronal (long) to transverse (short) vessels and the Longitudinal Index (Li) (n=4 grafts per group). FIG. 2D shows examples of blood vessel morphology from UBMs implanted on pig rectus muscles with or without a barrier (10× magnification). CD31 highlights blood vessels within rectus grafts at 2 weeks post-implant; counterstained with hematoxylin or methyl green.

FIG. 3 shows a representative Study Design. Phase 1 is shown in the left panel. Phase 2 is shown in the right panel.

FIGS. 4A-4D show early central perfusion and long-term regeneration of smooth muscle in RM-UBMs transplanted to the bladder. FIG. 4A shows an image of an RM-UBM sewn to the bladder after partial cystectomy. FIG. 4B shows quantification of the sizes of the grafts at the time of transplant and two weeks later at harvest; graphed as a percentage of size maintained (n=3 per group); t-test (*=p<0.05). FIG. 4C shows histologic analyses of RM-UBM and UBM bladder transplants at 2 weeks. H&E, CD31 (vascularity), SMA (smooth muscle) and panCK (urothelium) stains are shown for various areas within augmented bladders. Protein detection via Vector Red chromogen with methyl green counterstain. Arrows identify blue ink-perfused blood vessels in native bladder, UBM near anastomosis and central RM-UBM. FIG. 4D shows morphologic differences between UBM and RM-UBM after 3 months on the bladder. H&E stain, 10× magnification.

### DEFINITIONS

All ranges disclosed herein include the endpoints of the range, subranges, and any values in between to the first significant digit. For example, a range of 1 to 10 includes the subranges 1 to 9, 2 to 10, 1-5, 5-10, etc., and the values 1.1 to 9.9, 1.2 to 9.8, etc.

The term “about”, when referring to a numerical value or range of numerical values, includes values that are plus or minus 10% of the numerical value, including +/−1%, 2%, 3%, 4%, 5%, 6%, 7%, 8%, 9% or 10% of the value or range of values modified by the term about.

The terms “coronal”, “coronally directed” “coronally oriented” or “long” blood vessels refer to blood vessels that are longer than the standard cross-section.

The term “decellularized” or “devitalized” tissue refers to tissue in which a majority, most or substantially all of the living cells have been removed from the tissue. However, even after decellularization, the “decellularized” tissue can still contain growth factors and angiogenic factors attached to the decellularized matrix tissue.

The term “acellular tissue” refers to a tissue in which substantially all of the living cells have been removed from the tissue.

The term “transverse”, “transversely directed” or “short” blood vessel refers to blood vessels with lengths equal to or less than the mean width of a vessel lumen (cross section).

The term “longitudinal index” (Li) refers to the “Sum of the lengths all LONG vessels” divided by the “Absolute number of all LONG and SHORT vessels.”

The term “pore size” refers to the diameter of the individual pores in a membrane. Pore size is typically specified in micrometers (μm). Most membranes contain a distribution of pore sizes. Nominal pore size ratings typically refer to the predominant pore size of a filtration media; pores larger and smaller than the nominal rating may be present. Absolute pore size ratings typically refer to the largest pore size of a membrane and it is expected that all pores will be equal to or smaller than the absolute rating.

The term “porosity” refers to the percent of the total surface area of the porous membrane occupied by the pores.

The term “subject” refers to an animal or mammal that is suitable for a tissue graft of the present disclosure. The terms “subject” and “patient” can be used interchangeably. The term includes rodents, domesticated pets (cats and dogs), livestock such as pigs, goats, sheep cows, and horses, and humans.

### DETAILED DESCRIPTION

The present disclosure provides compositions and methods for directing the growth of blood vessels in a tissue graft. The compositions and methods provide the advantage of directing the growth of long blood vessels from the periphery of the graft, which recapitulates the native blood vessel orientation in the host tissue. The compositions comprise a tissue graft that comprises a matrix attached to a barrier. The barrier comprises a porous membrane that permits passage of water and nutrients but blocks blood vessel growth. The methods comprise implanting the tissue graft on a host tissue in a subject and incubating the graft on the host tissue such that blood vessels grow around the edges (periphery) of the barrier and into the matrix. The methods can further comprise transplanting the vascularized tissue graft to a second location in the subject. Thus, the methods can be used in a staged implant procedure to create autologous, vascularized bioengineered tissue grafts.

Embodiments of the disclosure are described below.

### Tissue Grafts

In some embodiments, the compositions of the disclosure comprise a tissue graft. In some embodiments, the tissue graft comprises a matrix attached to a barrier. The individual components of the tissue graft are described below.

### Barrier

The barrier described herein, or a portion or region thereof, comprises a porous membrane that permits passage of water and nutrients through imbibition. In some embodiments, the porous membrane comprises a pore size that permits passage of water and nutrients, but the pore size is too small to allow cells required for angiogenesis to pass through the barrier. In some embodiments, the porous membrane blocks blood vessel growth (angiogenesis) through the barrier, such that blood vessel growth occurs from the peripheral edge of the barrier or graft but does not occur through a region of the barrier comprising the porous membrane. It will be understood that blood vessel growth can also occur through portions or regions of the barrier that do not comprise a porous membrane and are intentionally left open (membrane free) by design. Thus, in some embodiments, the barrier comprises internal openings in the porous membrane that permit blood vessel growth through the barrier, which results in a predetermined pattern of blood vessel growth.

In some embodiments, the porous membrane comprises In some embodiments, the porous membrane comprises a plurality of pores having pore sizes or diameters in the range of about 0.1 to about 5 microns, e.g., about 0.1 to about 1.0 microns, about 0.1 to about 2.0 microns, about 0.1 to about 3.0 microns, about 0.1 to about 4.0 microns, about 0.1 to about 5.0 microns; about 0.2 to about 1.0 microns, about 0.2 to about 2.0 microns, about 0.2 to about 3.0 microns, about 0.2 to about 4.0 microns, about 0.1 to about 5.0 microns; about 0.3 to about 1.0 microns, about 0.3 to about 2.0 microns, about 0.3 to about 3.0 microns, about 0.3 to about 4.0 microns, about 0.3 to about 5.0 microns; about 0.4 to about 1.0 microns, about 0.4 to about 2.0 microns, about 0.4 to about 3.0 microns, about 0.4 to about 4.0 microns, about 0.4 to about 5.0 microns; about 0.5 to about 1.0 microns, about 0.5 to about 2.0 microns, about 0.5 to about 3.0 microns, about 0.5 to about 4.0 microns, or about 0.5 to about 5.0 microns. In some embodiments, the pore size is about 1.0 microns to about 2.0 microns, about 1.0 to about 3.0 microns, about 1.0 to about 4.0 microns, about 1.0 to about 5.0 microns; about 2.0 to about 3.0 microns, about 2.0 to about 4.0 microns, about 2.0 to about 5.0 microns; about 3.0 to about 4.0 microns, about 3.0 to about 5.0 microns; or about 4.0 to about 5.0 microns. In some embodiments, the plurality of pores has a pore size or diameter ranging from about 0.1 to about 10.0 microns, e.g., about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0 or 10.0 microns.

In some embodiments, the pore size or diameter may vary in different areas of the porous membrane. Thus, in some embodiments, the porous membrane comprises a plurality of pores having different pore sizes or diameters in different, discrete or non-overlapping regions of the membrane. For example, in some embodiments, the porous membrane comprises a plurality of pores having a first pore size/diameter in a first region of the membrane, a second pore size/diameter in a second region of the membrane, a third pore size/diameter in a third region of the membrane, and so on. The pore sizes/diameters in the different regions of the porous membrane can be selected from the ranges and values above.

In some embodiments, the porous membrane comprises a plurality of pores having different pore sizes or diameters that are interspersed or distributed across the membrane or a region thereof. The plurality of pores having different pore sizes/diameters that are interspersed or distributed across the membrane or a region thereof can be selected from the ranges and values above.

In general, a minimum overall porosity of approximately 50%, along with a pore size of approximately 35-100 microns is considered optimal for blood vessel formation (Oliviero, O., Ventre, M., and Netti, P. A. (2012). Functional porous hydrogels to study angiogenesis under the effect of controlled release of vascular endothelial growth factor. Acta Biomater. 8, 3294-3301. doi: 10.1016/j.actbio.2012.05.019). Thus, in some embodiments, the pore size of the barrier membrane is less than the pore size required for blood vessel formation. In some embodiments, the nominal or average pore size is in the range of about 1 to 35 microns, e.g., about 1, 2, 3, 4, 5, 6, 7, 8, 9 10, 11, 12, 13, 14, 15, 16, 17, 18, 19, 20, 21, 22, 23, 24, 25, 26, 27, 28, 29, 30, 31, 32, 33, 34 or 35 microns. In some embodiments, the absolute pore size is equal to or less than about 35 microns, equal to or less than about 30 microns, equal to or less than about 25 microns, equal to or less than about 20 microns, equal to or less than about 15 microns, equal to or less than about 10 microns, or equal to or less than about 5, 4, 3, 2, or 1 microns.

In some embodiments, the porous membrane comprises a pore density in the range of about 1×10⁵ to 4×10⁸ pores/square cm (cm²), e.g., about 1×10⁵, 1×10⁶, 1×10⁷, 1×10⁸, 2×10⁵, 2×10⁶, 2×10⁷, 2×10⁸, 3×10⁵, 3×10⁶, 3×10⁷, 3×1, 4×10⁵, 4×10⁶, 4×10⁷, or 4×10⁸ pores/cm². It will be understood that pore density can vary with the pore size, such that barrier membranes having smaller diameter pore sizes can have greater pore density. In some embodiments, the pore size is 0.4 microns and the pore density is 2×10⁶ pores/cm².

In some embodiments, the porosity of the barrier membrane is less than that required for blood vessel growth through the membrane. Thus, in some embodiments, the porosity is less than 50% of the total surface area occupied by the pores. In some embodiments, the porosity ranges from less than 1% to less than 50% of the total surface area occupied by the pores. In some embodiments, the porosity is equal to or less than about 45%, 40%, 35%, 30%, 25%, 20%, 15%, 10%, 5%, 1% or 0.5% of the total surface area occupied by the pores.

In some embodiments, the porous membrane has a nominal thickness of about 5-15 microns, e.g., about 5, 6, 7, 8, 9, 10, 11, 12, 13, 14 or 15 microns. In some embodiments, the porous membrane has a nominal thickness of greater than or equal to 15 microns, e.g., about 15, 20, 25, 30, 35, 45, 50, 60, 70, 80, 90, 100, 150, 200, 250, 300, 350, 400, 450, 500, 600, 700, 800, 900 or 1000 microns. In some embodiments, the porous membrane has a nominal thickness of about 1 to 10 mm, e.g., about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10.0 mm.

### Materials of the Porous Membrane

The porous membrane can be made of any suitable, biocompatible material. In some embodiments, the porous membrane is a hydrophilic membrane. In some embodiments, the porous membrane is a synthetic membrane. Examples of synthetic porous membranes include membranes made of polyester, polyethene, polyethylene, polycarbonate, cellulose acetate, and nylon. In some embodiments, the porous membrane is a hydrophilic polyethylene terephthalate membrane. In some embodiments, the porous membrane is a polycarbonate membrane.

### Frame

The barrier can also include a frame or mesh that is attached to and supports and stabilizes the porous membrane. The frame can be made of any suitable, biocompatible material. For example, in some embodiments, the frame is made of silicone. In some embodiments, the frame is made of metal.

In some embodiments, the frame is made of a non-porous material. In some embodiments, the non-porous frame is attached to the edges of the porous membrane and comprises an internal opening that does not overlap the porous membrane.

In some embodiments, the frame is about 1 mm to 5 mm thick, e.g., about 1 mm, 2 mm, 3 mm, 4 mm or 5 mm thick.

The frame can have a variety of shapes depending on the type of tissue graft, for example, a square, rectangle, oval or circle and the location where the graft will be placed in the host subject.

In some embodiments, the frame is attached to the tissue graft. In some embodiments, one surface (proximal surface) of the frame is attached directly to the matrix material of the graft, and the porous membrane is attached to the opposite, distal surface of the frame. In some embodiments, the frame is attached to the porous membrane, and the porous membrane is located adjacent to the matrix material of the graft.

In some embodiments, the porous membrane is attached directly to the graft. In some embodiments, the porous membrane is attached directly to the matrix material of the graft.

### Matrix

The matrix of the tissue graft can be any suitable biocompatible and/or biodegradable material. In some embodiments, the matrix is a decellularized or devitalized matrix. In some embodiments, the matrix is an acellular matrix. In some embodiments, the matrix is a decellularized or acellular matrix derived from a mammalian or human tissue. In some embodiments, the matrix comprises decellularized tissue. In some embodiments, the decellularized tissue is selected from bladder, kidney, liver, heart, lung, pancreas, connective tissue, bone, epidermis, or dermis. In some embodiments, the matrix is decellularized or acellular urinary bladder matrix (UBM). In some embodiments, the matrix is decellularized or acellular porcine urinary bladder matrix (pUBM). A representative method for preparing UBM is provided in the Examples.

It will be understood that decellularized matrix tissue can still contain cellular debris and growth factors attached to the extracellular matrix. Thus, in some embodiments, the matrix comprises angiogenic or growth factors attached to the decellularized matrix material. In some embodiments, the matrix comprises decellularized tissue from a human, animal or plant source or organism.

In some embodiments, the matrix comprises decellularized or devitalized (i.e., acellular or substantially acellular) mammalian epithelial basement membrane. In some embodiments, the matrix comprises decellularized or devitalized mammalian epithelial basement membrane and a biotropic connective tissue such as the tunica propria. In some embodiments, the matrix comprises decellularized or devitalized epithelial basement membrane isolated from the urinary bladder. Suitable devitalized matrix materials and methods for producing the same are described in U.S. Pat. No. 6,576,265 (to Spievack).

In some embodiments, the matrix is a synthetic matrix. In embodiments where the matrix is a synthetic matrix, angiogenic and other growth factors can be attached to the synthetic matrix before implantation into the host tissue. In some embodiments, antibodies are attached to the synthetic matrix before implantation into the host tissue.

In some embodiments, the synthetic matrix in generated de novo. In some embodiments, the synthetic matrix comprises silk. In some embodiments, the synthetic matrix comprises collagen.

In some embodiments, the matrix comprises a natural or synthetic polymer. Suitable biocompatible synthetic and natural polymeric matrices are described in U.S. Pat. No. 7,569,076 (to Atala). In some embodiments, the natural or synthetic polymer is selected from collagen, cellulose ether, cellulose, cellulosic ester, fluorinated polyethylene, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, copolymers thereof, and physical blends thereof. In some embodiments, the matrix comprises a biodegradable polymer.

In some embodiments, the decellularized matrix comprises collagen. In some embodiments, the decellularized matrix comprises collagen and one or more of glycoproteins, proteoglycans, and/or glycosaminoglycans. In some embodiments, the decellularized matrix comprises collagen isolated from the submucosa of a vertebrate or mammal. Suitable acellular matrices are described in U.S. Pat. No. 7,771,717 (to Badylak et al.).

In some embodiment, the matrix of the tissue graft is implanted with the lumen-side of the matrix in contact with (or facing) the host tissue. In some embodiment, the matrix of the tissue graft is implanted with the serosa-side of the matrix in contact with (or facing) the host tissue. In some embodiments, the host tissue is a rectus muscle. In some embodiments, the matrix is a UBM, and the tissue graft is implanted with the lumen-side of the UBM in contact with (or facing) the host tissue. In some embodiments, the matrix is a UBM, and the tissue graft is implanted with the serosa-side of the UBM in contact with (or facing) the host tissue. In some embodiments, the matrix is a UBM, and the tissue graft is implanted with the lumen-side of the UBM in contact with (or facing) the rectus muscle. In some embodiments, the matrix is a UBM, and the tissue graft is implanted with the serosa-side of the UBM in contact with (or facing) the rectus muscle.

### Host Tissue

The tissue grafts of the disclosure can be implanted on a tissue within the body of a subject. Representative examples of where the tissue graft can be implanted in the body of a subject include any muscle in the body, in subcutaneous fat, or under the kidney capsule. Thus, in some embodiments, the host tissue is selected from a muscle, subcutaneous fat, or a kidney capsule. In some embodiments, the muscle is the rectus abdominis muscle.

In some embodiments, the tissue graft is about 0.1 cm² to about 500 cm² in area. For tissue grafts in human subjects, the tissue graft can be greater than 100 cm² in area, for example 100, 150, 200, 250, or 500 cm².

In some embodiments, the subject (host) is a mammal. In some embodiments, the subject is selected from a rodent (e.g., a mouse or rat), livestock (e.g., a pig), a companion animal (such as a dog or cat), or a human.

### Methods

The disclosure provides methods for directing the growth of blood vessels into a tissue graft.

In some embodiments, the method comprises the steps of i) implanting a graft on a tissue in a host organism, wherein the graft comprises a matrix attached to a barrier and the barrier is positioned between the host tissue and the matrix; and ii) incubating the graft in the host, wherein blood vessels grow around the edges or through internal openings of the barrier, thereby directing the growth of blood vessels into predetermined areas of the matrix.

In some embodiments, the method comprises a barrier as described herein. In some embodiments, the barrier comprises a porous membrane that permits passage of water and nutrients, for example, by imbibition (passive absorption of fluids) but blocks blood vessel growth. In some embodiments, barrier comprises a porous membrane with the characteristics described above. For example, in some embodiments, the porous membrane comprises a nominal or average pore size in the range of about 1 to 35 microns, e.g., about 1, 2, 3, 4, 5, 6, 7, 8, 9 10, 11, 12, 13, 14, 15, 16, 17, 18, 19, 20, 21, 22, 23, 24, 25, 26, 27, 28, 29, 30, 31, 32, 33, 34 or 35 microns. In some embodiments, the absolute pore size is equal to or less than about 35 microns, equal to or less than about 30 microns, equal to or less than about 25 microns, equal to or less than about 20 microns, equal to or less than about 15 microns, equal to or less than about 10 microns, or equal to or less than about 5, 4, 3, 2, or 1 microns. In some embodiments, pore size is 0.1, 0.2, 0.3, 0.4, 0.5, 0.6, 0.7, 0.8, 0.9, 1.0, 2.0, 3.0, 4.0, 5.0, 6.0, 7.0, 8.0, 9.0 or 10.0 microns.

In some embodiments, the porous membrane comprises a pore density in the range of about 1×10⁵ to 4×10⁸ pores/square cm (cm²), e.g., about 1×10⁵, 1×10⁶, 1×10⁷, 1×10⁸, 2×10⁵, 2×10⁶, 2×10⁷, 2×10⁸, 3×10⁵, 3×10⁶, 3×10⁷, 3×1, 4×10⁵, 4×10⁶, 4×10⁷, or 4×10⁸ pores/cm². In some embodiments, the pose size is 0.4 microns and the pore density is 2×10⁶ pores/cm².

In some embodiments, the porosity of the barrier membrane is less than that required for blood vessel formation. Thus, in some embodiments, the porosity is less than 50% of the total surface area occupied by the pores. In some embodiments, the porosity ranges from less than 1% to less than 50% of the total surface area occupied by the pores. In some embodiments, the porosity is equal to or less than about 45%, 40%, 35%, 30%, 25%, 20%, 15%, 10%, 5%, 1% or 0.5% of the total surface area occupied by the pores.

In some embodiments, the barrier comprises a porous polyester membrane.

In some embodiments, the barrier comprises a non-porous biocompatible mesh or frame attached to the porous membrane. The mesh or frame can function to stabilize and support the porous membrane, which is typically very thin (e.g., from about 5 to 15 microns thick). The mesh or frame can be about 1 to 2 mm thick. In some embodiments, the mesh or frame is made of or comprises silicone.

The barrier can comprise a single contiguous porous membrane that prevents blood vessel growth through the barrier. Alternatively, the barrier can comprise one or more openings that allow blood vessel growth through certain regions of the barrier but not others. This allows different patterns of blood vessel growth to infiltrate the tissue graft.

In some embodiments, the barrier is attached to a matrix described herein, such as a decellularized tissue matrix. The decellularized tissue can be selected from any suitable tissue, including bladder, kidney, liver or pancreas. In some embodiments, the matrix comprises decellularized UBM. In some embodiments, the matrix is a synthetic matrix. In some embodiments, the matrix is a synthetic matrix made from silk or collagen.

In some embodiments, the matrix comprises angiogenic or growth factors attached to the decellularized matrix material. In some embodiments, the matrix comprises decellularized tissue from a subject or patient that will receive the tissue graft (i.e., the graft is an autologous tissue graft).

In the methods, the tissue graft is implanted into or on a host tissue in a subject. The host tissue can be any suitable tissue. In some embodiments, the host tissue that receives the tissue graft is selected from muscle, subcutaneous fat, or a kidney capsule. In some embodiments, the muscle is the rectus abdominis muscle.

In some embodiments of the methods, the tissue graft is implanted such that the lumen-side of the matrix is in contact with (or facing) the host tissue. In some embodiments of the methods, the tissue graft is implanted such that the serosa-side of the matrix is in contact with (or facing) the host tissue. In some embodiments, the host tissue is a muscle. In some embodiments, the host tissue is a rectus muscle.

In some embodiments, the tissue graft is about 25 cm² to about 300 cm² in area. For tissue grafts in human subjects, the tissue graft can be greater than or equal to 100 cm² in area, for example greater than or equal to 100, 150, 200, 250, or 300 cm².

Following implantation in the host tissue, the graft is incubated for a period of time that allows blood vessels to grow around the edge of the barrier and/or through internal openings in the barrier into the matrix. In some embodiments, the tissue graft is implanted for a period of about 1 week to about 6 months. In some embodiments, the tissue graft is implanted for longer than 6 months. In some embodiments, the tissue graft size is greater than or equal 100 cm² and the tissue graft is implanted for a period of about 6 months or longer.

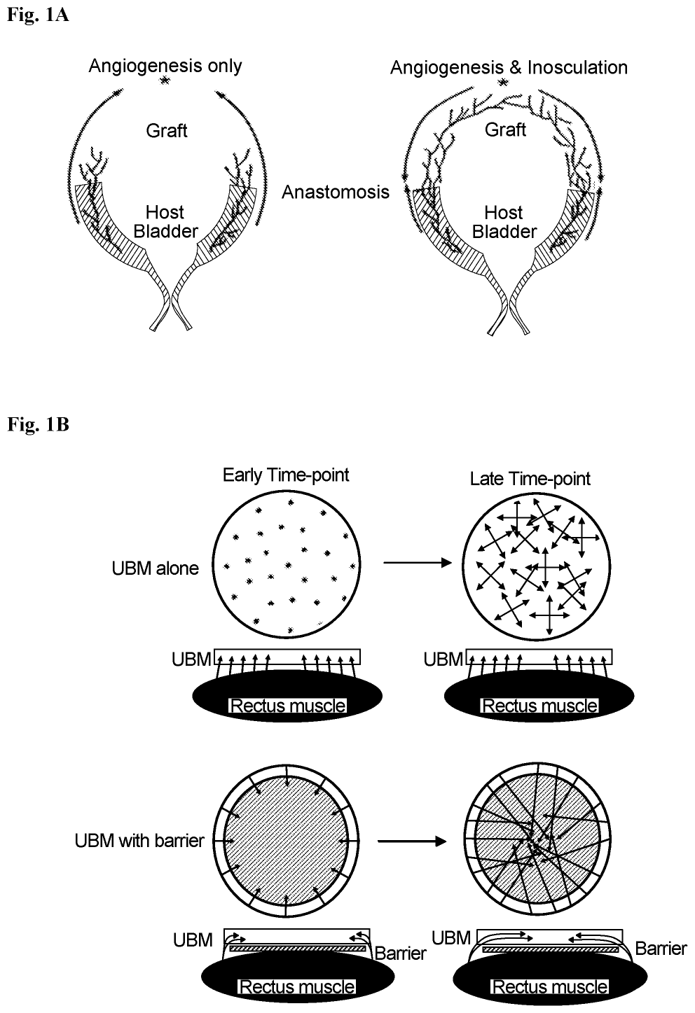

In some embodiments, following implantation in the host tissue, blood vessels grow around the edges of barrier and toward the center of the matrix, recapitulating native blood vessel orientation. The method produces long blood vessels that grow in the coronal plane of the graft. See FIGS. 1A and 1B, for example. Thus, in some embodiments, the average length of coronal blood vessels is increased compared to grafts without a barrier. In some embodiments, the ratio of coronal (long) to transverse (short) vessels is increased compared to grafts without a barrier. In some embodiments, the longitudinal index (Li) of blood vessels is increased compared to grafts without a barrier. This provides an advantage, as the coronal plane is the future site of anastomosis of the pre-vascularized graft following transplantation to a second tissue. In some embodiments, the blood vessels grow through intentional internal openings in the barrier to create a desired pattern of blood vessel growth.

In some embodiments, the vascularity of the graft, expressed as mean vessel density (MVD), increases over time, such that at earlier time points there will be fewer, longer vessels (lower MVD, high Li) in the center of the grafts with a barrier, but at later time points, the MVD will be equivalent between grafts with and without barriers. Thus, in some embodiments, the mean vessel density (MVD) is similar between grafts equal to or greater than 100 cm² that are implanted for about 6 months with and without a barrier.

In another aspect, the disclosure provides a method for producing a vascularized tissue graft. In some embodiments, the method comprises the steps of i) implanting a graft on a tissue in a host organism, wherein the graft comprises a matrix attached to a barrier and the barrier is positioned between the host tissue and the matrix; and ii) incubating the graft in the host such that blood vessels grow around the edges of the barrier or through internal openings. In some embodiments, the method produces blood vessels that have an increased longitudinal index in the center of the tissue graft compared to tissue grafts without a barrier. In some embodiments, the blood vessels have an increased longitudinal index in the coronal plane of the graft.

### Second Transplantation

Following implantation in the host tissue for a period of time that allows blood vessels to grow in an intentional pattern in the matrix, the vascularized graft (referred to as a “barrier-matured graft” or “pre-vascularized graft”) can be transplanted to a second location or second host tissue in the subject.

The second host tissue can be any tissue in the subject in need of a tissue graft. For example, the second host tissue can be damaged or diseased tissue. In some embodiments, the second host tissue is selected from heart, kidney, urinary bladder, spinal cord covering, liver, gastrointestinal tract tissues such as stomach, small intestine, or large intestine, pancreas, lung, and dermal or epidermal tissue. The methods and compositions of the present disclosure can induce growth of endogenous tissues including epithelial and connective tissues when target tissues are placed in contact with the tissue grafts described herein in vivo. In some embodiments, the second host tissue is urinary bladder tissue.

In some embodiments, the graft is implanted in the first host tissue for a period of about 1 week to 6 months before being transplanted to the second host tissue. In some embodiments, the period of time between the first tissue graft implantation and transplantation to the second host tissue is greater than or equal to 6 months, e.g., 6, 7, 8, 9, 10, 11, 12 or more months. In some embodiments, the period of time between the first tissue graft implantation and transplantation to the second host tissue is greater than or equal to one year, e.g., 1, 1.5, 2, 2.5, 3. 3.5, 4, 4.5, 5 or more years.

The following Examples describe exemplary embodiments of the disclosure.

### EXAMPLES

### Example 1

This example describes a representative method for preparing urinary bladder matrix (UBM).

### Preparation of UBMs.

Pig bladders are harvested from USDA-grade pigs. The bladders are decellularized using a protocol adapted from several published protocols.³⁴⁻³⁶ The optimized protocol takes pig bladders (distended and with agitation) through three sequential steps of hypotonic solution, Triton detergent-based hypertonic solution, and RNAse/DNAse solution. Pig UBMs are then sterilized using neutralized peracetic acid and washed thoroughly in sterile water.

### Example 2

This example describes a representative method for transplanting a tissue graft onto the rectus abdominis muscle.

### Rectus Muscle Implantation.

UBMs are prepared as 12 cm² grafts. Four (4) pigs are anesthetized according to IACUC protocol at the UC Davis Surgical Research Facility (SRF). For rectus implantation, a midline incision is made through the lower abdomen skin, and the anterior rectus fascia is opened sagittal and elevated off the anterior aspect of the rectus muscle. Grafts are implanted on the anterior rectus muscle and secured to the muscle using 4-0 polypropylene suture; 6 grafts (3 on either side of the midline) are implanted per pig. Grafts are placed with the either the lumen-side (n=3 per pig; 6 total) or the serosa-side (n=3 per pig; 6 total) of the UBM facing the rectus muscle. The abdomen are closed in 4 layers; muscle, fascia, subcutaneous space and skin, followed by surgical glue on the incision. Animals are given analgesics and antibiotics during the post-op period, according to standard veterinary practices. Two pigs are euthanized at each of 3 and 6 months.

### RM-UBM Harvest and Tissue Processing.

Pigs are anesthetized, and nitroglycerin (8 ug/kg) and sterile India ink (65 ml/kg blood volume) are given i.v. and allowed to circulate for 10-15 minutes. The purpose of India ink perfusion is to assess the function of newly formed blood vessels within grafts after harvest (by histology). Immediately after India ink perfusion, euthanasia is induced while under deep general anesthesia. Grafts are identified, measured and harvested from the anterior rectus, then fixed in 10% neutral buffered formalin. Tissues are processed for paraffin-embedding and sectioned for immunohistochemical (IHC) analyses.

### Pig RM-UBM Sampling and Analyses.

As needed for statistical significance, UBMs are evaluated for each orientation and at each of 2 time-points (3 and 6 months). Random samples from each RM-UBM are evaluated for histology by H&E staining and for the expression of CD31 and SMA for vascularization and smooth muscle formation, respectively. IHC is used to assess both the quality and quantity of vascularization and muscle formation. Quantification of staining is performed using ImageJ (NIH), where areas of interest (protein markers) are highlighted using the Thresholding tool and the Analyze Particles function are used to calculate staining indices, giving stain per area of tissue as a percentage.¹⁸ Graft blood vessels are stained for CD31, from which mean vessel density (MVD) is calculated as a percentage. The vessels will also be assessed for maturation. The presence of pericytes and smooth muscle via NG2 and alpha-smooth muscle actin (SMA) staining, respectively, are measured within vessels (CD31) and presented as an index. Smooth muscle presence is evaluated by SMA staining and the architecture of the muscle (bundles) are evaluated via H&E. Observation of colored ink within CD31+ blood vessels is evidence of perfusion and thereby function. A minimum of n=3 analyzable grafts per condition is expected to be sufficient to observe statistically significant differences in protein expression and vascularization within grafts¹⁸.

### Example 3

This example describes the growth of blood vessels in tissue grafts comprising a barrier of the disclosure.

The inventors have shown that host bladder vessels grow into the graft quickly and then anastomose with graft vessels (inosculation). Therefore, it is important that engineered graft vessels are organized and poised to function prior to bladder transplantation.¹⁸ Unlike other transplantation sites where there is a large surface area of contact between graft and host (e.g., skin), transplants to the bladder are limited to contact along the peripheral edge of the graft (FIG. 1A). The central portion of the graft is far beyond the reach of early imbibition and angiogenesis. The bladder also lacks a single artery and vein that can be directly anastomosed to the host blood supply upon transplant. Thus, pre-existing vessels that are long and coronally-oriented from the anastomosis toward the central portion of the graft are paramount to establish blood supply throughout the graft via inosculation (FIG. 1B).

The goal of the experiments is to create a graft with long and radially oriented vessels that will facilitate earlier perfusion to the center of the graft upon transplantation to the bladder.

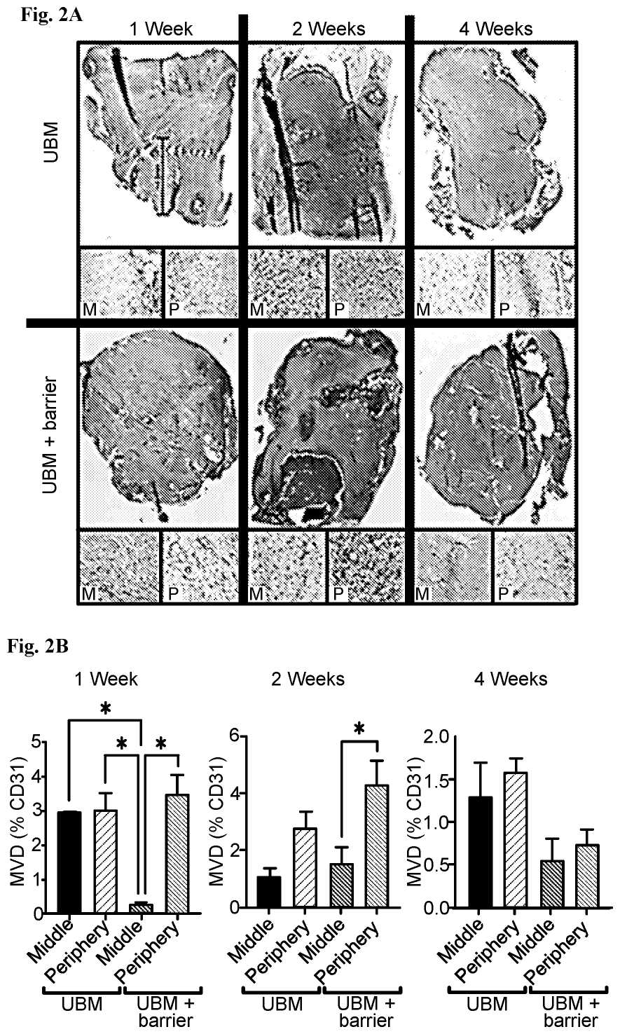

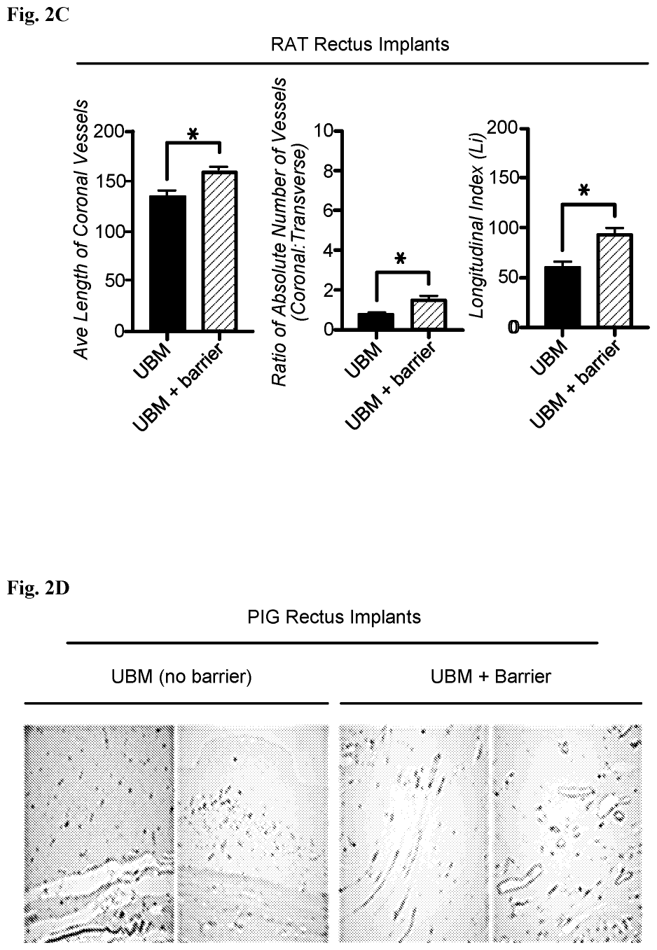

Plastic surgeons have investigated the use of different types of barriers between skin flaps and the host bed to induce ischemia and mediate imbibition.³⁸ Barriers can be used to block vascularization of flaps. This “preconditioning” has been shown to lead to more viable flaps.³⁹ A barrier was tested with a pore size that allows imbibition but prevents cellular migration in the rat rectus implantation model. The goal was to create an environment where angiogenesis from the rectus muscle would only occur from the peripheral edge but at the same time allow hydration through imbibition. This induces long vessels in the coronal plane. Histology at one week showed that grafts with barriers had peripheral vascularization and cellularization (FIGS. 2A, 2B). The central areas were essentially free of cells but appeared to be hydrated grossly without contraction. Later time points (2 and 4 weeks) showed central cellularization comparable to grafts without barriers and equivalent vascularization based upon MVD (surface area) (FIG. 2B). Based on CD31 staining, the length of individual vessels was measured using ImageJ. Blood vessels with lengths equal to or less than the mean width of a vessel lumen (cross section) were counted as “transversely directed”, or SHORT. Vessels that were longer than the standard cross-section were manually marked and referred to as “coronally directed”, or LONG. This data was used to calculate the average length of long vessels, the ratio of LONG:SHORT vessels and the longitudinal index, which is defined as:

$${L}_{i} = \frac{Sum of the lengths all LONG vessels}{Absolute number of all LONG and SHORT vessels} .$$

The three indices in FIG. 2C demonstrate the presence of longer vessels in UBMs with barriers compared to those without at 2 weeks. However, at 4 weeks post-implant, the length of vessels in grafts with and without barriers was equivalent, possibly due to small graft size (1.8 cm²) or eventual branching. The presence of dye and red blood cells in vessels implied patency and flow (data not shown). Rectus grafts with barriers were also tested in the pig model and preliminary data shows more elongated vessels in the presence of the barrier (FIG. 2D).

To determine if the barrier technique works in the pig with larger grafts, two different experiments with increasing graft size and time for regeneration were designed. The large grafts (100 cm²) and 6-month time point reflect expected size and time in a clinical setting.

### Study Design [6-18 Months]

Preparation of UBMs. UBMs (25 cm² or 100 cm²) are prepared as noted in Example 1. Barriers (polyester membranes—0.4 micron pore size, 2×10⁶ pores/cm²; Sterlitech) are secured to UBMs with ligating clips and using implantable mesh as a stabilizing backbone.

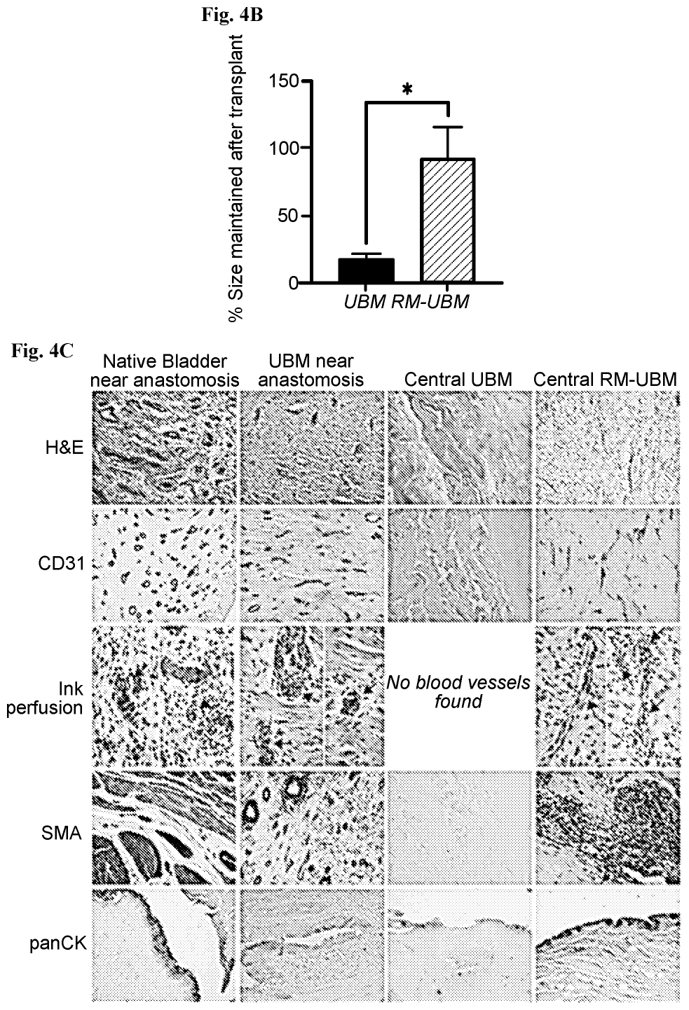

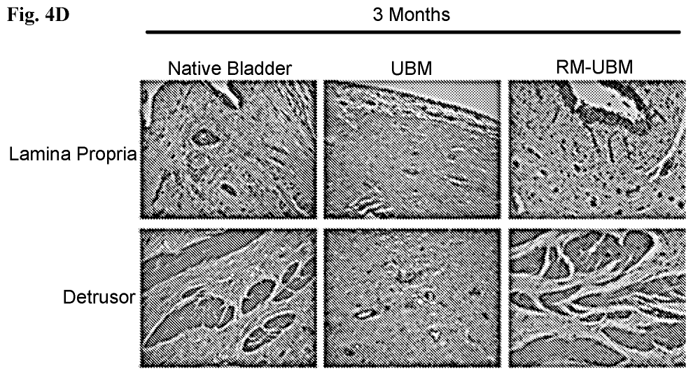

Implantation of UBMs+/−barrier onto the rectus muscle. The barrier technique is tested in 2 phases to determine vascular patterning (see FIG. 4).

### Phase 1:

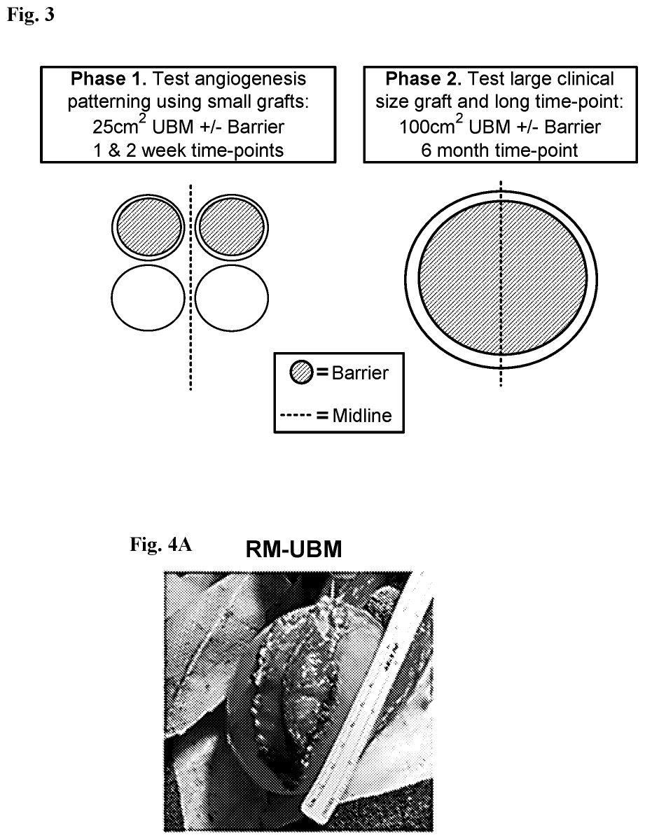

In this phase, two pigs per time-point are used to evaluate vascularization of UBMs+/−barrier in vivo. These grafts are 25 cm², implanted 4 per pig (n=2 no barrier; n=2 with barrier) and harvested at 2-week and 1-month time-points. This will yield n=4 grafts per condition at each timepoint. (FIG. 3, left panel)

### Phase 2:

In this phase, larger grafts (100 cm²; 1 per pig) are implanted to evaluate clinical-size grafts. A 100 cm² UBM is implanted alone or with a barrier. Four pigs per condition (8 pigs total) will yield n=4 grafts per condition at 6 months. (FIG. 3, right panel)

Harvest of grafts. At 1 and 2 weeks post-implant, pigs are anesthetized and given heparin intravenously to reduce clotting. The inferior epigastric vessels are exposed and cannulated and Microfil, a radiopaque colored compound that can occupy vascular spaces, is injected, which perfuses the rectus muscle and grafts, then cured within vessels post-mortem. The pig is euthanized while under deep sedation. Immediately post-mortem, the grafts are measured for size, harvested and then imaged by radiography. Fresh strips are frozen for biomechanical studies. Tissue will also be fixed and processed for histologic analyses.

Radiology for vessel orientation. To achieve gross visualization of the pattern of graft vasculature, radiographic imaging is used. Images are taken using a Faxitron Cabinet X-ray System.

Histology and Immunohistochemistry. Tissue sections are stained and analyzed for vascularity (MVD) and smooth muscle formation as detailed in Aim 1. Grafts are assessed for vessel length using the Li.

Biomechanical testing. Methods as described in Aim 1 and compared to UBM and native bladder tissue.

### Expected Outcomes:

1. For the 25 cm² grafts, the early time points (1 and 2 weeks) will show decreased central MVD with a barrier.
2. Although a higher Li for small grafts (rat) was not observed at 4 weeks, it is expected that the 100 cm² grafts (1-month time point), will show a higher Li in the central graft that was blocked by the barrier. Due to the large graft size, the barrier will lead to a lower central MVD but a higher Li.
3. For the 100 cm² grafts at 6-month time point, equivalent MVD throughout the graft between those with and without barriers is expected. On the other hand, the grafts with barriers will have longer vessels in the coronal plane based on radiographic imaging and Li quantification. Thus, a barrier will induce generation of long vessels from the periphery in the coronal plane that recapitulates native bladder vessel orientation.
4. For all graft sizes and experiments, central cellularization is lessened by the barrier in early time points but long-term time points will demonstrate similar muscle regeneration with and without a barrier.
5. Biomechanics and contractility is similar between grafts grown with or without a barrier. However, grafts are stiffer and less elastic, but stronger than native bladder tissue.

### Example 4

This example describes exemplary vascularization and regeneration of tissue grafts after transplantation to the pig bladder.

Tissue grafts with barriers were prepared as described above. Large (100 cm²) UBMs were implanted on the rectus muscle. The rectus-matured grafts were harvested at 2 months. A portion of the bladder dome, equivalent to the graft size, was excised and the graft was then sewn into place similar to augmentation techniques (FIG. 4A). Three control pigs per timepoint had bladder wall replacement with UBMs. Upon harvest two weeks later, RM-UBMs had maintained 90% of their surface area size after transplant whereas control UBMs maintained only 20% of their size (FIG. 4B). The bladder was harvested after administration of systemic India ink and evaluated by IHC (FIG. 4C). In the control animals, angiogenesis was limited to the peripheral edges of the grafts (i.e., UBM near anastomosis) without any evidence of central graft perfusion; central graft contraction was also evident. On the other hand, autologous RM-UBMs had early central graft perfusion at 2 weeks, moderately advanced muscle formation and no signs of tissue degradation. By 3 months, RM-UBM graft morphology resembles native bladder with a highly vascularized lamina propria and luminal surface lined by multi-layered urothelium. Muscle formation is also much more advanced in RM-UBMs, demonstrating muscle bundle formation (FIG. 4D). UBM grafts had no evidence of bundle formation and instead exhibited a very homogenous cellularization lacking obvious differentiation and with little to no urothelium on the luminal surface.

The data presented in this example supports the potential clinical use of this staged implant procedure as a way to create autologous, vascularized bioengineered bladder tissue that can be harvested from the rectus muscle and attains central perfusion within two weeks after transplant to the bladder.

### REFERENCES

1. Kurzrock, E. A. & Polse, S. Renal deterioration in myelodysplastic children: urodynamic evaluation and clinical correlates. J Urol 159, 1657-1661 (1998).
2. El-Azab, A. S. & Moeen, A. M. The satisfaction of patients with refractory idiopathic overactive bladder with onabotulinumtoxinA and augmentation cystoplasty. Arab J Urol 11, 344-349 (2013).
3. Kurzrock, E. A., Baskin, L. S. & Kogan, B. A. Gastrocystoplasty: long-term followup. J Urol 160, 2182-2186 (1998).
4. McDougal, W. S. Metabolic complications of urinary intestinal diversion. J Urol 147, 1199-1208 (1992).
5. Tu, D. D. et al. Bladder tissue regeneration using acellular bi-layer silk scaffolds in a large animal model of augmentation cystoplasty. Biomaterials 34, 8681-8689 (2013).
6. Coutu, D. L., Mahfouz, W., Loutochin, O., Galipeau, J. & Corcos, J. Tissue engineering of rat bladder using marrow-derived mesenchymal stem cells and bladder acellular matrix. PLOS One 9, e111966 (2014).
7. Roelofs, L. A. J. et al. Bladder Regeneration Using Multiple Acellular Scaffolds with Growth Factors in a Bladder. Tissue Eng Part A 24, 11-20 (2018).
8. Pokrywczynska, M. et al. Understanding the role of mesenchymal stem cells in urinary bladder regeneration-a preclinical study on a porcine model. Stem Cell Res Ther 9, 328 (2018).
9. Affas, S. et al. Augmentation Cystoplasty of Diseased Porcine Bladders with Bi-Layer Silk Fibroin Grafts. Tissue Eng Part A 25, 855-866 (2019).
10. Leonhäuser, D. et al. Two differentially structured collagen scaffolds for potential urinary bladder augmentation: proof of concept study in a Göttingen minipig model. J Transl Med 15, 3 (2017).
11. Rossetto, V. J. V. et al. Grafts of porcine small intestinal submucosa seeded with cultured homologous smooth muscle cells for bladder repair in dogs. Acta Vet Scand 55, 39 (2013).
12. Zhang, Y., Frimberger, D., Cheng, E. Y., Lin, H.-K. & Kropp, B. P. Challenges in a larger bladder replacement with cell-seeded and unseeded small intestinal submucosa grafts in a subtotal cystectomy model. BJU Int 98, 1100-1105 (2006).
13. Chua, M. E., Farhat, W. A., Ming, J. M. & McCammon, K. A. Review of clinical experience on biomaterials and tissue engineering of urinary bladder. World J Urol 38, 2081-2093 (2020).
14. Atala, A., Bauer, S. B., Soker, S., Yoo, J. J. & Retik, A. B. Tissue-engineered autologous bladders for patients needing cystoplasty. Lancet 367, 1241-1246 (2006).
15. Joseph, D. B., Borer, J. G., De Filippo, R. E., Hodges, S. J. & McLorie, G. A. Autologous cell seeded biodegradable scaffold for augmentation cystoplasty: phase II study in children and adolescents with spina bifida. J Urol 191, 1389-1395 (2014).
16. Cheng, E. Y. Editorial comment. J Urol 191, 1395 (2014).
17. Clark, E. R. & Clark, E. Microscopic observations on the growth of blood capillaries in the living mammal. undefined (1939).
18. Osborn, S. L., So, M., Hambro, S., Nolta, J. A. & Kurzrock, E. A. Inosculation of blood vessels allows early perfusion and vitality of bladder grafts—implications for bioengineered bladder wall. Tissue Eng Part A 21, 1906-1915 (2015).
19. Osborn, S. L. and Kurzrock, E. A. Autologous regeneration of blood vessels in urinary bladder matrices provides early perfusion after transplant to the bladder. (2021).
20. Kikuno, N. et al. Nerve growth factor combined with vascular endothelial growth factor enhances regeneration of bladder acellular matrix graft in spinal cord injury-induced neurogenic rat bladder. BJU Int 103, 1424-1428 (2009).
21. Loai, Y. et al. Bladder tissue engineering: tissue regeneration and neovascularization of HA-VEGF-incorporated bladder acellular constructs in mouse and porcine animal models. J Biomed Mater Res A 94, 1205-1215 (2010).
22. Sharma, A. K. et al. Cotransplantation with specific populations of spina bifida bone marrow stem/progenitor cells enhances urinary bladder regeneration. Proc Natl Acad Sci USA 110, 4003-4008 (2013).
23. Baranski, J. D. et al. Geometric control of vascular networks to enhance engineered tissue integration and function. Proc Natl Acad Sci USA 110, 7586-7591 (2013).
24. Koffler, J. et al. Improved vascular organization enhances functional integration of engineered skeletal muscle grafts. Proc Natl Acad Sci USA 108, 14789-14794 (2011).
25. Chen, X. et al. Rapid anastomosis of endothelial progenitor cell-derived vessels with host vasculature is promoted by a high density of cotransplanted fibroblasts. Tissue Eng Part A 16, 585-594 (2010).
26. Chen, X. et al. Prevascularization of a fibrin-based tissue construct accelerates the formation of functional anastomosis with host vasculature. Tissue Eng Part A 15, 1363-1371 (2009).
27. Ko, I. K. et al. Bioengineered transplantable porcine livers with re-endothelialized vasculature. Biomaterials 40, 72-79 (2015).
28. Song, J. J. et al. Regeneration and experimental orthotopic transplantation of a bioengineered kidney. Nat Med 19, 646-651 (2013).
29. Ott, H. C. et al. Regeneration and orthotopic transplantation of a bioartificial lung. Nat Med 16, 927-933 (2010).
30. Ott, H. C. et al. Perfusion-decellularized matrix: using nature's platform to engineer a bioartificial heart. Nat Med 14, 213-221 (2008).
31. Schultheiss, D. et al. Biological vascularized matrix for bladder tissue engineering: matrix preparation, reseeding technique and short-term implantation in a porcine model. J Urol 173, 276-280 (2005).
32. Yamataka, A. et al. Living-related partial bladder transplantation for bladder augmentation in rats: an experimental study. J Pediatr Surg 38, 913-915 (2003).
33. Campbell, G. R. et al. The peritoneal cavity as a bioreactor for tissue engineering visceral organs: bladder, uterus and vas deferens. J Tissue Eng Regen Med 2, 50-60 (2008).
34. Song, L. et al. Bladder acellular matrix and its application in bladder augmentation. Tissue Eng Part B Rev 20, 163-172 (2014).
35. Bolland, F. et al. Development and characterisation of a full-thickness acellular porcine bladder matrix for tissue engineering. Biomaterials 28, 1061-1070 (2007).
36. Yang, B. et al. Development of a porcine bladder acellular matrix with well-preserved extracellular bioactive factors for tissue engineering. Tissue Eng Part C Methods 16, 1201-1211 (2010).
37. Fyhrie, D. P. & Christiansen, B. A. Bone Material Properties and Skeletal Fragility. Calcif Tissue Int 97, 213-228 (2015).
38. Jones, M. et al. Influence of recipient-bed isolation on survival rates of skin-flap transfer in rats. J Reconstr Microsurg 17, 653-658; discussion 659 (2001).
39. Tatlidede, S. et al. A novel murine island skin flap for ischemic preconditioning. J Surg Res 154, 112-117 (2009).
40. Tanaka, S. T., Thangappan, R., Eandi, J. A., Leung, K. N. & Kurzrock, E. A. Bladder wall transplantation—long-term survival of cells: implications for bioengineering and clinical application. Tissue Eng Part A 16, 2121-2127 (2010).
41. Thangappan, R., Eandi, J. A., Modi, J. & Kurzrock, E. A. Epithelium-free bladder wall graft: epithelial ingrowth and regeneration—clinical implications for partial cystectomy. J Urol 187, 1450-1457 (2012).
42. Bochner, B. H. et al. A serous lined antireflux valve: in vivo fluorourodynamic evaluation of antireflux continence mechanism. J Urol 160, 112-115 (1998).

All references, issued patents, patent applications and patent publications cited herein are hereby incorporated by reference in their entirety, for all purposes.

What is claimed is:

1. A method for directing the growth of blood vessels in a tissue graft, comprising the steps of
(i) implanting a graft on a tissue in a host organism, wherein the graft comprises a matrix attached to a barrier and the barrier is positioned between the host tissue and the matrix; and
(ii) incubating the graft in the host, wherein blood vessels grow around the edges of the barrier, thereby directing the growth of blood vessels around the barrier and into the matrix.

2. A method for producing a vascularized tissue graft, comprising the steps of
i) implanting a graft on a tissue in a host organism, wherein the graft comprises a matrix attached to a barrier and the barrier is positioned between the host tissue and the matrix; and
ii) incubating the graft in the host such that blood vessels grow around the edges of the barrier and have an increased longitudinal index in the center of the tissue graft compared to tissue grafts without a barrier.

3. The method of claim 1 or 2, wherein the barrier comprises a porous membrane that permits passage of water and nutrients but blocks blood vessel growth.

4. The method of any one of claims 1 to 3, wherein the porous membrane comprises a pore size from about 0.1 micron to about 5 microns.

5. The method of claim 4, wherein the porous membrane comprises a pore size of about 0.4 microns.

6. The method of any one of claims 3 to 5, wherein the porous membrane is a polyester membrane.

7. The method of any one of claims 3 to 6, wherein the barrier comprises a non-porous biocompatible stabilizing mesh or frame attached to the porous membrane.

8. The method of claim 7, wherein the stabilizing mesh or frame is about 1 mm to about 2 mm thick.

9. The method of claim 7 or 8, wherein the stabilizing mesh or frame comprises silicone.

10. The method of any one of claims 1 to 9, wherein the barrier comprises a single contiguous porous membrane.

11. The method of any one of claims 1 to 10, wherein the barrier comprises one or more internal openings that permit blood vessel growth through the barrier.

12. The method of claim 1, wherein the host tissue is selected from the group consisting of a muscle, subcutaneous fat, and a kidney capsule.

13. The method of claim 12, wherein the muscle is a rectus abdominis muscle.

14. The method of any one of claims 1 to 13, wherein the matrix comprises decellularized tissue.

15. The method of claim 14, wherein the decellularized tissue is selected from the group consisting of bladder, kidney, liver, heart, lung, pancreas, placenta membrane, connective tissue, bone, epidermis, and dermis.

16. The method of claim 15, wherein the matrix comprises decellularized urinary bladder matrix (UBM).

17. The method of any one of claims 1 to 13, wherein the matrix is a synthetic matrix.

18. The method of any one of claims 1 to 17, wherein the graft size is about 0.1 cm² to about 500 cm².

19. The method of any one of claims 1 to 18, wherein the graft is implanted in the host tissue for a period of about 2 weeks to about 6 months.

20. The method of any one of claims 1 to 19, wherein the average length of coronal blood vessels is increased compared to grafts without a barrier.

21. The method of any one of claims 1 to 20, wherein the ratio of coronal (long) to transverse (short) vessels is increased compared to grafts without a barrier.

22. The method of any one of claims 1 to 21, wherein the longitudinal index (Li) of blood vessels is increased compared to grafts without a barrier.

23. The method of any one of claims 1 to 22, wherein the mean vessel density (MVD) is similar between grafts equal to or greater than 100 cm² that are implanted for about 6 months with and without a porous barrier.

24. The method of any one of claims 1 to 23, further comprising transplanting the graft to a second host tissue after a period of time sufficient for blood vessel growth into the matrix.

25. The method of claim 24, wherein the second host tissue is selected from the group consisting of heart, kidney, urinary bladder, spinal cord, liver, gastrointestinal tract tissues such as stomach, small intestine, or large intestine, pancreas, lung, and dermal or epidermal tissue.

26. The method of claim 25, wherein the second host tissue is bladder tissue.

27. The method of any one of claims 24 to 26, wherein the period of time is from about 2 weeks to about 6 months.

28. A tissue graft comprising a matrix attached to a barrier, the barrier comprising a porous membrane, and the matrix comprising a decellularized tissue.
